(19) United States
(12) Patent Application Publication
(10) Pub. No.: US 2018/0292674 A1
(43) Pub. Date: Oct. 11, 2018

(54) EYEGLASS CUSHIONING DEVICE AND METHOD

(71) Applicant: Ryan Edward Bond, Calgary (CA)

(72) Inventor: Ryan Edward Bond, Calgary (CA)

(21) Appl. No.: 15/480,115

(22) Filed: Apr. 5, 2017

(51) Int. Cl. G02C 5/14; G02B 27/01; A61F 9/02

(52) U.S. Cl. CPC G02C 5/143; A61F 9/026; G02B 27/017

(57) ABSTRACT

An eyeglass cushioning device useful for diminishing pressure placed upon a user's ears and surrounding area through extended headphone, goggle, protective eyewear and circumaural headset use. The eyeglass cushioning device includes a sleeve member, at least one headset spacer and a cushioning pad. The sleeve member is flexible and configured to slide over the temple of a user-wearer's eyeglasses. The headset spacer is attached to the sleeve member, and may be comprised of an upper and lower spacer relative to the eyeglass temple, configured to minimize deformation of the ear, and thereby diminish discomfort of a user-wearer with extended headset use. The cushioning pad is a compliant material configured to wrap around the back of the ear of a user-wearer during headphone, headset, protective eyewear or goggle use.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of support structures for electrical audio and/or video signal processing systems and devices, and more specifically relates to an eyeglass cushioning device and method.

### 2. Description of Related Art

Many people require vision correction. Arguably more than 60% of adults wear prescription glasses today. Further, use of personal audio or video electronics such as headphones, augmented reality goggles, and video game headsets has greatly increased in the recent past. Many users of personal electronics worn about the head also wear eyeglasses, sunglasses or similar at the same time. Workers in occupations requiring the use of personal protective equipment may need to wear prescription glasses in combination with protective eyewear, in combination with a hardhat. When these types of items are worn together by a user-wearer for an extended period of time, the combination of the eyeglass temple and the temple tip can become uncomfortable to the user over time due to pressure applied to the ear through use of headphones, headsets, safety glasses or goggles. As a result, the user may experience pain behind and around the ear. Therefore, there is a significant need for an apparatus to enable a user to comfortably wear audio headphones, video or gaming headsets and the like, as well as eyewear simultaneously for extended periods.

U.S. Pat. No. 8,454,158 to Glenn Joseph Dillard relates to a device for coupling a wireless headset to an eyeglass frame. The described device for coupling a wireless headset to an eyeglass frame includes an apparatus to facilitate wearing eyewear and a wireless headset at the same time. The apparatus may include a first tubular element that may be configured to be slipped over a temple of the eyewear. The apparatus may also include a second tubular element that may be configured to receive an around the ear securing element coupled with, or included with, the wireless headset.

U.S. Pub. No. 2008/0205683 to Frank Michael Weyer discloses a method and apparatus for attaching earphones to eyeglass and similar frames. The apparatus is comprised of an earphone including an elongated support arm that supports the earphone electronics and speaker unit. In one or more embodiments, the elongated support arm has a configuration such that it has a first free end, an elongated, typically curved center portion, and a second end to which the earphone speaker unit is attached. An expandable, resilient, tubular sleeve having a bore is slid over the free end such that it is disposed along the center portion. The combination of the earphone and resilient sleeve forms an audio unit that may be attached to a temple of an eyeglass frame by sliding the free end of the temple through the resilient sleeve until the free end of the temple projects from the rear of the sleeve.

None of the above inventions and patents, taken either singly or in combination, is seen to describe the invention as claimed. Thus, a need exists for a reliable eyeglass cushioning device and method, and to avoid the above-mentioned problems.

### BRIEF SUMMARY OF THE INVENTION

In view of the foregoing disadvantages inherent in the known art comprising ear structures for audio and/or video devices, the present disclosure provides a novel eyeglass cushioning device and method. The general purpose of the present disclosure, which will be described subsequently in greater detail, is to provide a device, system and method for use with headphones, augmented or virtual reality goggles, gaming headsets, circumaural headsets, safety glasses, protective eyewear, and the like which supports and cushions the ear and thereby provides greater comfort to a user with headset or protective eyewear use for extended periods of time. The device may provide support and cushioning to the ear of a user-wearer regardless of whether eyeglass use is required. One non limiting example of this is a pair of protective eyewear or safety glasses having eyeglass cushioning devices attached before, during or after fabrication and prior to use by a user-wearer. A second non limiting example is circumaural headphones or headsets where use of the device is not limited to a user-wearer's need for glasses.

The eyeglass cushioning device disclosed herein may include a sleeve member, upper and lower headset spacers relative to the eyeglass temple when worn by the user-wearer, and a cushioning pad located in alignment with the lower headset spacer; the cushioning pad being fixed to and extending away from the sleeve outer surface.

The sleeve member may be comprised of a first material and configured to attach to an eyeglass temple; the sleeve member having first and second sleeve ends opposite one another, and a sleeve axis extending between the first and second sleeve ends. Correctly positioned, a temple tip of a user's eyeglasses may slide through an opening in a first sleeve end of the sleeve member, emerging from an opening in a second sleeve end. The sleeve member also may have a sleeve inner surface defining a cavity and further configured to interface with the eyeglass temple, and a sleeve outer surface to which the headset spacers and cushioning pad are fixed.

The upper and lower headset spacers may be comprised of a second material, and further may be attached on opposite sides of the sleeve member relative to the eyeglass temple, extending substantially parallel to the sleeve member. The lower headset spacer may also be attached to a portion of an outer surface of the sleeve member and in alignment with the cushioning pad. The headset spacers may serve to prevent deformation of a user's ears and relieve pressure created on the eye glass arm and as a result, provide greater comfort to a user.

The cushioning pad of the eyeglass cushioning device may be comprised of a third material, and is fixed to at least a portion of and extending away from the sleeve outer surface. The cushioning pad is further configured to wrap behind the ear of the user-wearer, and may have a curvature conforming to a back of the ear of the user-wearer when worn.

According to another embodiment of the invention, use of the eyeglass cushioning device may be achieved as a system for relieving pressure from a head mounted device. The system may be comprised of left and right eyeglass cushioning devices including left and right sleeve members made of a first material and configured to attach to left and right eyeglass temples of a user-wearer's eyeglasses, left and right headset spacer elements made of a second material and further attached to and extending substantially parallel to the left and right sleeve members, and left and right cushioning pads made of a third material, fixed to and extending away from the left and right sleeve outer surfaces, the cushioning pads configured to wrap behind the ears of the user-wearer when worn. The system configured to diminish pressure on the ears of the user-wearer when worn in conjunction with the head mounted device, such as a headset, virtual reality goggles, safety glasses, protective eyewear or circumaural headphones.

According to yet another embodiment of the invention, use of the eyeglass cushioning device may be achieved as a method for relieving pressure from a head mounted device, comprising the steps of sliding an eyeglass temple tip of a pair of eyeglasses through a first opening in a first end of the sleeve member such that an end of the temple tip emerges from an opening in a second end in the sleeve member, placing the pair of eyeglasses with the eyeglass cushioning device attached, upon a user-wearer's ears, positioning the eyeglass cushioning device such that the cushioning pad wraps behind a user's ear and provides pressure relief for a user-wearer's ear during use of the head mounted apparatus, and placing the head mounted apparatus, such as a circumaural headphone, gaming headsets or goggles, safety glasses, or protective eyewear upon a user-wearer's ears and positioning the cushioning pad between the head of the user-wearer and the head mounted apparatus.

In other embodiments, connection between the left and right cushioning devices may be made through use of a cord or wire attached to an end of each eyeglass cushioning device. An alternate means for connecting the left and right eyeglass cushioning devices may be made by way of a flexible strap which connects on either end to the devices and encircles the back of a user's head when in use.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an eyeglass cushioning device and method, constructed and operative according to the teachings of the present disclosure.

FIG. 1 is a perspective view of the eyeglass cushioning device during an ‘in-use’ condition showing the device in place behind the ear on a user's eyeglasses, according to an embodiment of the disclosure.

FIG. 2 is a perspective view of the eyeglass cushioning device of FIG. 1, in a ‘ready-for-use’ condition according to an embodiment of the present disclosure.

FIG. 3A is a perspective view of the eyeglass cushioning device of FIG. 1, as installed on a user's eyeglasses, according to an embodiment of the present disclosure. Further illustrated is a cross sectional plane through the device as shown in 3B.

FIG. 3B is a cross sectional view of the section of the eyeglass cushioning device as specified in FIG. 3A, according to an embodiment of the present disclosure.

FIG. 4 is a posterior view of a user's head having the eyeglass cushioning device in place upon a user-wearer's eyeglasses, as specified in FIG. 1.

FIG. 5 is a flow diagram illustrating a method of use for the eyeglass cushioning device, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to support structures for electrical audio and/or video signal processing systems and devices, and more particularly to an eyeglass cushioning device and method as used to improve user comfort when extended use of headphones, circumaural headsets, safety glasses, protective eyewear or goggles is desired, and a user also wears prescription glasses, sunglasses or the like.

Generally, the eyeglass cushioning device may comprise a sleeve member made of a first material and configured to attach to an eyeglass temple; the sleeve member having a first sleeve end, a second sleeve end opposite the first sleeve end; a sleeve axis extending between the first sleeve end and the second sleeve end; a sleeve inner surface configured to interface with the eyeglass temple; and a sleeve outer surface through which an eyeglass temple tip may be passed. The eyeglass cushioning device also has at least one headset spacer fixed to the sleeve outer surface, and configured to diminish pressure placed on an ear of a user-wearer and on the eyeglass temple by and during use of a headset.

The eyeglass cushioning device also preferably has a cushioning pad fixed to and extending away from the sleeve outer surface; the cushioning pad configured to wrap behind the ear of the user-wearer when worn. The headset spacers and cushioning pad may serve to reduce pressure placed upon the ear and surrounding areas through extended headphone, goggles and headset use. It may be appreciated that a gradation in pressure reduction may be achieved. For example, it is anticipated that pressure reductions ranging from 10% to 60% may be possible, dependent upon materials selection and design. It is conceivable that in some instances 100% pressure relief may be attainable wherein the headphones rest solely upon the headset spacers and cushioning pad, and do not contact the ear of a user-wearer.

The present disclosure describes an eyeglass cushioning device which slides over the temple tip of a user-wearer's glasses and onto the temple of the glasses to enable comfortable and extended use of headsets, goggles, protective eyewear or headphones. The device has a sleeve member made of a first material, having a first sleeve end, a second sleeve end, an outer surface, an inner surface; wherein the inner surface defines a cavity sufficient to accommodate at least one of an eyeglass temple and an eyeglass temple tip. The eyeglass temple tip is placed through an opening near the first sleeve end, and subsequently may emerge through an opening proximal to the second sleeve end. The sleeve member may be comprised of any flexible, durable material that will conform to the shape of the eyeglass temple. Non limiting examples may be neoprene, silicone, or any rubber composition having compliance such that it conforms to the shape of the eyeglass temple.

Attached in a direction substantially parallel to the sleeve axis and fixed to the outer surface is at least one headset spacer made of a second material and configured to diminish pressure placed on an ear of a user-wearer and on the eyeglass temple by and during use of a headset. The headset spacer is configured to have a thickness greater than that of the combined thickness of the sleeve member and the cross sectional thickness of the eyeglass temple. The headset spacer may be comprised of one or more headset spacers attached in a direction parallel with the sleeve axis and opposing each other, wherein an upper headset spacer relative to the eyeglass temple may span an entire length of the outer surface of the sleeve member. A bottom headset spacer is aligned parallel with the cushioning pad, relative to the eyeglass temple, and may span at least a portion of the length of the outer surface of the sleeve member. In other configurations, the bottom headset spacer may be completely replaced by the cushioning pad, extending substantially from the first sleeve end to the second sleeve end of the sleeve member.

Both upper and lower headset spacers are solid throughout, and comprised of materials having lower compliance than the sleeve member, such as rubber compositions having greater rigidity, one example is higher elastic modulus silicones or suitably equivalent material. The headset spacers are configured to diminish pressure placed on an ear of a user-wearer and on the eyeglass temple by and during use of a headset, headphones, goggles, or similar. In this manner, pressure from use of personal electronics worn about the head is transferred from the eyeglass temple piece to the headset spacers and cushioning pad collectively, allowing for greater user comfort with extended headset use. It should be noted that headset as defined herein includes, but is not limited to, a headphone, virtual or augmented reality goggles, a single piece earphone, a dual piece earphone, circumaural headphones, or similar.

Fixed to and extending away from the sleeve outer surface is a cushioning pad made of a third material. An end of the cushioning pad is proximate to a first end of the sleeve member and extends substantially parallel to the axis of the sleeve member. The cushioning pad is configured to wrap behind the ear of the user-wearer when worn. The cushioning pad is located opposite the upper headset spacer relative to the eyeglass temple, and in alignment with a bottom headset spacer. The cushioning pad preferably has a curvature conforming to a back of the ear of the user-wearer when worn. During use, the curvature of the cushioning pad conforms to a back of the ear of the user-wearer is configured facing towards and in contact with a user's ear to distribute pressure applied by audio headphones, augmented reality goggles, circumaural headsets or other electronic devices worn about the head. The cushioning pad is configured to wrap behind the ear of the user-wearer when worn. The cushioning pad further may be comprised at least in part of silicone, a gel, memory foam or pressure sensitive foam, or any compliant rubber suitable to be in contact with human skin over extended periods.

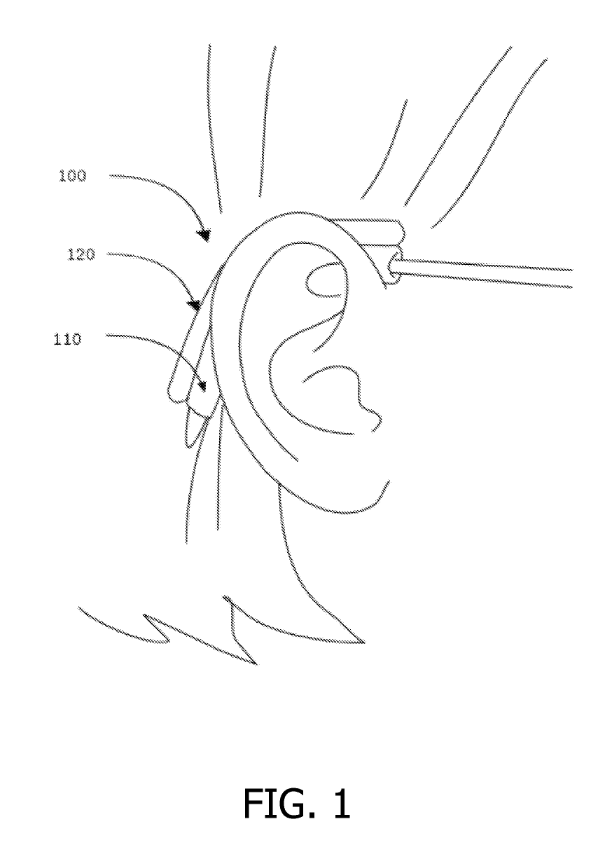

Referring now more specifically to the drawings by numerals of reference, there is shown in FIGS. 1-4, various views of an eyeglass cushioning device, 100. Referring now to FIG. 1 which shows an eyeglass cushioning device, 100 during an ‘in-use’ condition, according to an embodiment of the present disclosure. Here, the eyeglass cushioning device, 100 may be beneficial for use by a user to diminish pressure placed upon the ear and surrounding area through extended use of a headset or protective eye wear, as mentioned previously.

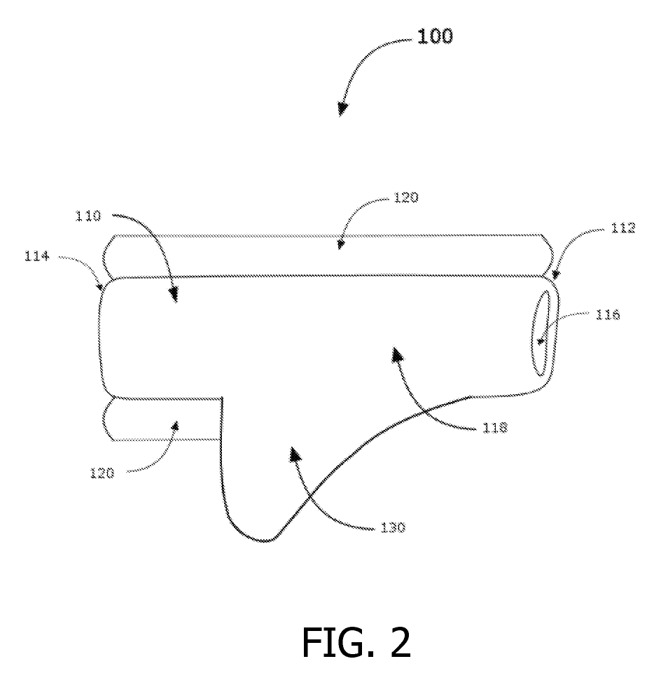

As illustrated, the eyeglass cushioning device 100 may include a sleeve member (110), and a headset spacer (120) upon which pressure from external apparatus worn about the head is placed. The eyeglass temple of a user's eyeglasses is illustrated enclosed in the sleeve member (110). Referring now to FIG. 2, the eyeglass cushioning device, 100 is shown in a ‘ready-for-use’ condition wherein the sleeve axis is shown in a linear configuration, and includes a sleeve member (110) having a first end (112), a second end (114), a sleeve inner surface (116), a sleeve outer surface (118), at least one headset spacer (120), and a cushioning pad (130). The sleeve member (110) is sufficiently flexible to conform to a shape of an eyeglass temple tip or temple when manually installed on the eyeglass temple tip during use. The at least one headset spacer (120) is shown here with both upper and lower headset spacers, relative to the eyeglass temple, depicted. The cushioning pad (130) may be fabricated from a variety of compliant materials, such as a gel, silicone, or memory foam, which provide support for a user's ear during use of headphone, headset, protective eye wear, or other head mounted apparatus.

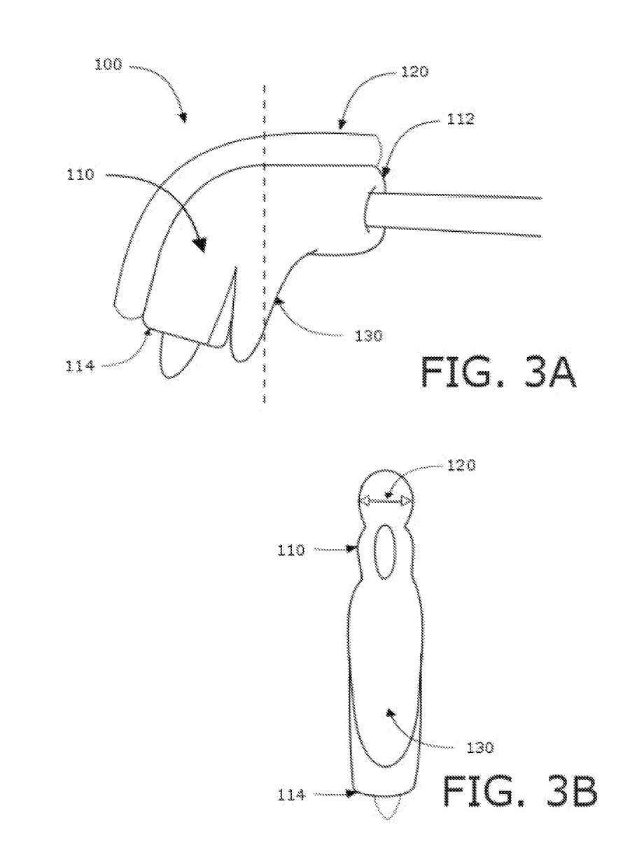

Referring now to FIG. 3A, a perspective view of the eyeglass cushioning device 100 of FIG. 1 is depicted; wherein the eyeglass cushioning device is shown as placed upon a user's eyeglasses, according to an embodiment of the present disclosure. As depicted, the sleeve element (110) conforms to the shape of the user's temple tip, resulting in the sleeve axis assuming a curved configuration. A vertical indicator line through the eyeglass cushioning device shows where the cross sectional view illustrated in FIG. 3B is taken.

Referring now to FIG. 3B, the upper headset spacer (120), relative to the eyeglass temple, is visible at the top of the drawing in a cross sectional view of the eyeglass cushioning device of FIG. 3A. The upper headset spacer (120) is shown to have a greater width than that of the cross sectional thickness of the temple and sleeve member (110). Also illustrated in cross section is the cushioning pad (130), and the eyeglass temple tip protruding from an opening in the second end (114) of the sleeve member. It may be appreciated that the actual length of the cushioning pad may vary with design, according to an embodiment of the present disclosure.

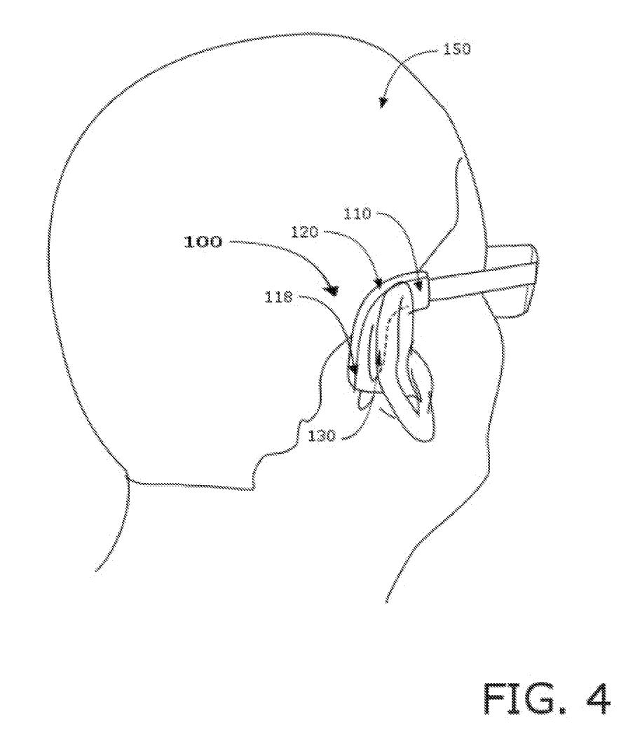

Referring now to FIG. 4, shown is a posterior view of a user's head (150), illustrating the eyeglass cushioning device (100) located in a proper position to diminish pressure applied to the ears during headphone or headset use. The upper headset spacer (120), relative to the eyeglass temple, may be viewed across an outer surface (118) of the sleeve member (110). The location of the cushioning pad (130) is illustrated in place behind the ear, according to an embodiment of the present disclosure.

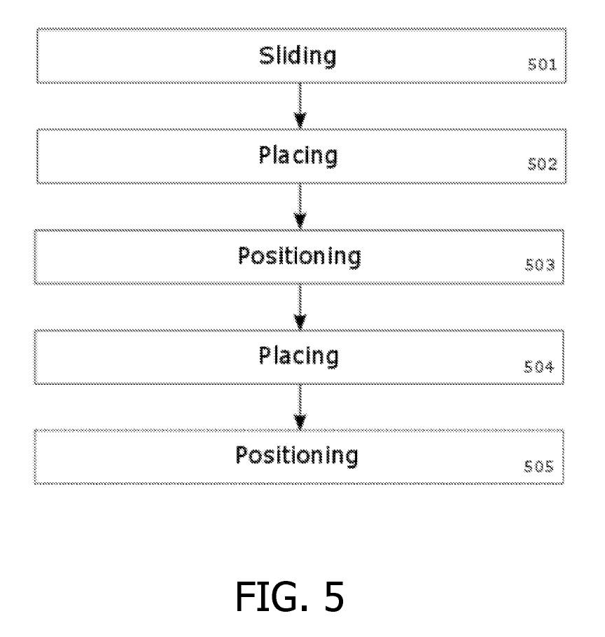

FIG. 5 is a flow diagram 550 illustrating a method for using 500 an eyeglass cushioning device 100 to enhance user comfort, according to an embodiment of the present disclosure. In particular, the method for using 500 an eyeglass cushioning device to enhance user comfort, 100 may include one or more components or features of the eyeglass cushioning device 100 as described above. As illustrated, the method for using 500 an eyeglass cushioning device 100 to enhance user comfort may include the steps of: step one (501), sliding an eyeglass temple tip through an opening in a first end of a sleeve member such that the end of the temple tip emerges from an opening in a second end in the sleeve member; step two (502), placing the eyeglasses with the eyeglass cushioning device installed, upon a user wearer's ears; step three (503), positioning the eyeglass cushioning device such that the cushioning pad wraps around and behind a user's ear and provides pressure reduction for the ear during headphone, headset, protective eyewear or goggle use; step four (504), placing headphones, goggles, headsets, protective eyewear or similar upon a user's ears and over the eyeglass cushioning device; and step five (505), positioning the cushioning pad between the head of the user-wearer and the head mounted apparatus.

According to another embodiment of the invention, use of the eyeglass cushioning device may be achieved as system for relieving pressure from a head mounted device. The system may comprise a left eyeglass cushioning device (100) including a left sleeve member (110) made of a first material and configured to attach to a left eyeglass temple, the left sleeve member having a first left sleeve end, a second left sleeve end opposite the first left sleeve end, and a left sleeve axis extending between the first left sleeve end and the second left sleeve end. The left sleeve member may also have a left sleeve inner surface configured to interface with the left eyeglass temple, a left sleeve outer surface, and at least one left headset spacer (120) fixed to the left sleeve outer surface, further configured to diminish pressure placed on a left ear of a user-wearer and on the left eyeglass temple by and during use of the head mounted device. The system may also be comprised of a left cushioning pad (130) fixed to and extending away from the left sleeve outer surface, the left cushioning pad configured to wrap behind the left ear of the user-wearer when worn. The system may further be comprised of a right eyeglass cushioning device (100) including a right sleeve member (110) made of a first material and configured to attach to a right eyeglass temple, the right sleeve member having a first right sleeve end, a second right sleeve end opposite the first right sleeve end, and a right sleeve axis extending between the first right sleeve end and the second right sleeve end. The right sleeve member may also have a right sleeve inner surface configured to interface with the right eyeglass temple, a right sleeve outer surface, and at least one right headset spacer (120) fixed to the right sleeve outer surface, further configured to diminish pressure placed on a right ear of a user-wearer and on the right eyeglass temple by and during use of the head mounted device. The system may also be comprised of a right cushioning pad (130) fixed to and extending away from the right sleeve outer surface, the right cushioning pad configured to wrap behind the right ear of the user-wearer when worn.

In other embodiments, connection between the left and right cushioning devices may be made through use of a cord or wire attached to the second end of each device. An alternate means for connecting the left and right devices may be made by way of a flexible strap which connects on both strap ends to second ends of the left and right devices and encircles the back of a user's head when in use.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for using an eyeglass cushioning device to enhance user comfort (e.g., different step orders within above-mentioned list, elimination or addition of certain steps, including or excluding certain maintenance steps, etc.), are taught herein.

It should be noted that step 505 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for an eyeglass cushioning device (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An eyeglass cushioning device comprising:
a sleeve member made of a first material and configured to attach to an eyeglass temple, the sleeve member having a first sleeve end, a second sleeve end opposite the first sleeve end, a sleeve axis extending between the first sleeve end and the second sleeve end, a sleeve inner surface configured to interface with the eyeglass temple, and a sleeve outer surface; and
at least one headset spacer fixed to the sleeve outer surface, and configured to diminish pressure placed on an ear of a user-wearer and on the eyeglass temple by and during use of a head mounted apparatus.

2. The eyeglass cushioning device of claim 1, wherein said at least one headset spacer includes an upper headset spacer and a lower headset spacer, relative to the eyeglass temple when worn by the user-wearer.

3. The eyeglass cushioning device of claim 1, wherein said at least one headset spacer extends substantially parallel to the axis of the sleeve member.

4. The eyeglass cushioning device of claim 1, wherein the at least one headset spacer is made of a second material, the first material being more compliant than the second material.

5. The eyeglass cushioning device of claim 1, wherein the outer sleeve surface defines a sleeve thickness, said sleeve thickness measured perpendicular to the sleeve axis; and
wherein the at least one headphone headset spacer has greater thickness than the sleeve thickness.

6. The eyeglass cushioning device of claim 1, wherein the sleeve member includes a flexible tube.

7. The eyeglass cushioning device of claim 1, further comprising a cushioning pad fixed to and extending away from the sleeve outer surface, the cushioning pad configured to wrap behind the ear of the user-wearer when worn.

8. The eyeglass cushioning device of claim 7, wherein the cushioning pad is at least partially made of memory foam.

9. The eyeglass cushioning device of claim 7, wherein the cushioning pad is at least partially made of silicone.

10. The eyeglass cushioning device of claim 7, wherein the cushioning pad is at least partially made of a gel.

11. The eyeglass cushioning device of claim 1, wherein the head mounted apparatus is a pair of safety glasses or protective eye wear.

12. The eyeglass cushioning device of claim 7, wherein the cushioning pad has a curvature conforming to a back of the ear of the user-wearer when worn.

13. The eyeglass cushioning device of claim 1, wherein the head mounted apparatus is a circumaural headset or a virtual reality headset.

14. The eyeglass cushioning device of claim 7, wherein the cushioning pad is fixed to at least a portion of and including an entire length of the sleeve outer surface extending substantially from the first sleeve end to the second sleeve end of the sleeve member.

15. The eyeglass cushioning device of claim 7, wherein the cushioning pad is made of a third material.

16. The eyeglass cushioning device of claim 1, wherein the sleeve member is sufficiently flexible to conform to a shape of an eyeglass temple tip when manually installed on the eyeglass temple tip.

17. The eyeglass cushioning device of claim 1, wherein the sleeve member is at least partially made of neoprene or silicone.

18. The eyeglass cushioning device of claim 1, wherein the sleeve inner surface defines a cavity sufficient to accommodate at least one of an eyeglass temple and an eyeglass temple tip.

19. A system for relieving pressure from a head mounted device, the system comprising:
a left eyeglass cushioning device including
a left sleeve member made of a first material and configured to attach to a left eyeglass temple, the left sleeve member having a first left sleeve end, a second left sleeve end opposite the first left sleeve end, a left sleeve axis extending between the first left sleeve end and the second left sleeve end, a left sleeve inner surface configured to interface with the left eyeglass temple, and a left sleeve outer surface; and
at least one left headphone headset spacer fixed to the left sleeve outer surface, and configured to diminish pressure placed on a left ear of a user-wearer and on the left eyeglass temple by and during use of the head mounted device,
a left cushioning pad fixed to and extending away from the left sleeve outer surface, the left cushioning pad configured to wrap behind the left ear of the user-wearer when worn; and
a right eyeglass cushioning device including
a right sleeve member made of a first material and configured to attach to a right eyeglass temple, the right sleeve member having a first right sleeve end, a second right sleeve end opposite the first right sleeve end, a right sleeve axis extending between the first right sleeve end and the second right sleeve end, a right sleeve inner surface configured to interface with the right eyeglass temple, and a right sleeve outer surface,
at least one right headphone headset spacer (120) fixed to the right sleeve outer surface, and configured to diminish pressure placed on a right ear of a user-wearer and on the right eyeglass temple by and during use of head mounted device, and
a right cushioning pad (130) fixed to and extending away from the right sleeve outer surface, the right cushioning pad configured to wrap behind the right ear of the user-wearer when worn.

20. A method for relieving pressure from a head mounted device, the method comprising the steps of:
providing an eyeglass cushioning device including a sleeve member configured to attach to an eyeglass temple, and further including a cushioning pad fixed to and extending from an outer surface of the sleeve member, the cushioning pad configured as a support between a head of a user-wearer and the head mounted device;
sliding an eyeglass temple tip of a pair of eyeglasses through a first opening in a first end of the sleeve member such that an end of the temple tip emerges from an opening in a second end in the sleeve member;
placing the pair of eyeglasses with the eyeglass cushioning device attached, upon a user-wearer's ears;
positioning the eyeglass cushioning device such that the cushioning pad rests behind a user's ear and provides pressure relief for a user-wearer's ear during use of the head mounted device;
placing the head mounted device upon a user-wearer's ears and over the eyeglass cushioning device; and
positioning the cushioning pad between the head of the user-wearer and the head mounted apparatus.
